(19) United States
(12) Patent Application Publication
(10) Pub. No.: US 2009/0326338 A1
(43) Pub. Date: Dec. 31, 2009

(54) METHOD AND APPARATUS FOR ANALYZING GAS COMPONENT DERIVED FROM LIVING BODY AND DISEASE DETERMINATION SUPPORTING APPARATUS

(75) Inventors: Naofumi Kobayashi, Tokyo (JP); Sunao Takeda, Tokyo (JP); Naoki Kobayashi, Tokyo (JP)

Correspondence Address: KIMBLE INTELLECTUAL PROPERTY LAW, PLLC, 1701 PENNSYLVANIA AVE., NW, SUITE 300, WASHINGTON, DC, 20006, US

(73) Assignee: NIHON KOHDEN CORPORATION, Tokyo (JP)

(21) Appl. No.: 12/495,750

(22) Filed: Jun. 30, 2009

(30) Foreign Application Priority Data
Jun. 30, 2008 (JP) 2008-171226

(51) Int. Cl. A61B 5/00

(52) U.S. Cl. 600/300

(57) ABSTRACT

A method of analyzing a gas component derived from living body, includes: extracting a first gas component from a gas contained in an atmosphere by a first method and removing the extracted first gas component from the gas to defecate the atmosphere; obtaining a mixed gas of the defecated atmosphere and a second gas component from a subject; extracting the first gas component from the mixed gas by a second method; and analyzing the first gas component extracted from the mixed gas. The second method is the same as the first method.

### BACKGROUND OF THE INVENTION

The present invention relates to an apparatus for analyzing a gas component derived from living body, and more particularly to an apparatus for analyzing a gas component derived from living body which is preferably used for analyzing a gas generated from an expired gas, blood, urine, skin or the like of a subject in the case of, for example, a clinical or health examination in the medical fields a check for drink drive, or drug enforcement.

There is the following related-art apparatus as a sampling apparatus for a gas (expired gas) derived from living body (see JP-A-9-126958). Hereinafter, a gas derived from living body is often referred to as a living-body derived gas.

FIG. 9 is a view showing the configuration of the gas sampling apparatus for a living-body derived gas (expired gas). The apparatus includes: an expired gas sampling mask 11 which is to cover the mouth and the nostrils; clean-air supplying unit 12 for supplying clean air to the expired gas sampling mask 11; a clean-air flow path 13 through which the clean-air supplying unit 12 communicates with the expired gas sampling mask 11; and an expired gas flow path 14 which guides the air in the expired gas sampling mask 11 toward an expired gas analyzing apparatus N.

The clean-air flow path 13 and the expired gas flow path 14 are coupled to the expired gas sampling mask 11 through a ribless valve 15. The ribless valve 15 functions in the same manner as the case where a check valve is disposed in each of the flow paths 14, 15.

A clean-air storage buffer 16 is disposed in the middle of the clean-air flow path 13. An expired gas storage buffer 17 is disposed downstream from the expired gas flow path 14. The expired gas flow path 14 is coupled to the expired gas analyzing apparatus N through the expired gas storage buffer 17. The reference numeral 18 denotes an exhaust air flow path through which an excess expired gas in the expired gas storage buffer 17 is exhausted to the open air.

The portions shown in FIG. 9 will be described in detail. The expired gas sampling mask 11 is formed into a shape which is suitable for covering the mouth and nostrils of the subject S, such as a bowl shape, and formed by a flexible material, so that the closeness to the face of the subject S is improved. According to the configuration, during sampling of the expired gas, the expired gas can be prevented from being mixed with the open air, and hence only the purer expired gas can be supplied to the expired gas analyzing apparatus N.

The clean-air supplying unit 12 previously supplies clean air into the expired gas sampling mask 11. The clean-air supplying unit 12 includes: a tank which is filled with clean air at a high pressure; pressure adjusting unit for reducing the pressure of the high-pressure clean air to the vicinity of the atmospheric pressure; and flow amount adjusting unit for supplying a constant amount of the pressure-reduced clean air toward the expired gas sampling mask 11.

The clean air in the tank is the air from which impurities are removed away, and which is purified so that the composition ratios of oxygen, nitrogen, and the like have a predetermined value. The composition ratios are referred in the future analysis of the expired gas.

The clean air supplied from the clean-air supplying unit 12 is supplied to the expired gas sampling mask 11 through the clean-air flow path 13. The clean-air flow path 13 is an elastic tube having an inner diameter of about 15 to 20 [mm]. The clean-air storage buffer 16 is disposed in the middle of the flow path.

The clean-air storage buffer 16 is a container having a capacity corresponding to plural respirations of the subject S. The capacity is set to about 5 liters. Usually, the volume of one respiratory inhalation of a human is about 400 to 500 [cc]. In the clean-air storage buffer 16, the capacitor is set to a value corresponding to about ten respirations.

Before sampling of the expired gas, the clean-air storage buffer 16 is in a state where it is filled with the clean air which is previously supplied from the clean-air supplying unit 12. In a state where the expired gas sampling mask 11 is attached to the subject S, therefore, the subject can sufficiently inhale the clean air stored in the clean-air storage buffer 16.

As an example of the clean-air supplying unit 12 in FIG. 9, there is the following related-art air defecating apparatus (see JP-A-2001-245987).

JP-A-2001-245987 discloses an oxygen and high-purity air supply system including a compressor; an oxygen concentrating system configured by a plurality of zeolite filling tank inlet two-way selector valves connected to the compressor, a plurality of zeolite filling tanks connected to the zeolite tank inlet two-way selector valves, respectively, and a plurality of zeolite filling tank outlet check valves connected to the zeolite tilling tanks, respectively; and a high-purity air system configured by an air filter connected to the compressor, and a plurality of high-purity air valves connected to the air filter (for example, claim 1 of JP-A-2001-245987).

Referring to FIG. 9 disclosed in JP-A-9-126958, in the related-art expired gas sampling apparatus, the air for respiration of the subject 5 is supplied by the clean-air supplying unit 12 from which impurities are previously removed away. A to-be-measured gas component derived from living body has a concentration of about ppb (0.0000001%). Therefore, the apparatus has a problem in that it requires much labor to produce clean air.

Even when the high-purity air supply system disclosed in JP-A-2001-245987 is employed as the clean-air supplying unit of the expired gas sampling apparatus disclosed in JP-A-9-126958, it is impossible to remove all impurities. In the case where the sensitivity of analyzing unit coincides with a component which cannot be removed by the clean-air supplying unit, there is a problem in that an analysis result of a gas component derived from living body is adversely affected.

### SUMMARY

It is therefore an object of the invention to provide a method and apparatus for analyzing a gas component derived from living body which is less affected by the impurity removal performance of clean-air supplying unit (air defecating unit), and in which analysis of a gas component derived from living body can be performed in a reduced number of steps.

In order to achieve the object, according to the invention, there is provided a method of analyzing a gas component derived from living body, the method comprising:

extracting a first gas component from a gas contained in an atmosphere by a first method and removing the extracted first gas component from the gas to defecate the atmosphere;

obtaining a mixed gas of the defecated atmosphere and a second gas component from a subject;

extracting the first gas component from the mixed gas by a second method; and

analyzing the first gas component extracted from the mixed gas,

wherein the second method is the same as the first method.

According to the invention, there is also provided an apparatus for analyzing a gas component derived from living body, the apparatus comprising:

a first trapping unit, which extracts a first gas component from a gas contained in an atmosphere by a first method, and removes the first gas component from the gas to defecate the atmosphere;

a mixing unit, which mixes the defecated atmosphere with a second gas component from a subject, to generate a mixed gas;

a second trapping unit, which extracts the first gas component from the mixed gas by a second method; and

a analyzing unit, which analyzes the first gas component extracted by the second trapping unit,

wherein the second method is the same as the first method.

The first trapping unit may cool the gas contained in the atmosphere, which flows thereinto, to liquefy or solidify the gas, thereby extracting the first gas component. The second trapping unit may cool the mixed gas, which flows thereinto, to liquefy or solidify the mixed gas, thereby extracting the first gas component.

Each of the first and second trapping units may be provided with a material which sorbs the first gas component.

The mixing unit may generate the mixed gas by allowing the subject to breathe the atmosphere defecated by the first trapping unit.

The mixing unit may generate the mixed gas by exposing a product substance of the subject or a part of the subject to the atmosphere defecated by the first trapping unit.

The first trapping unit may include a function which is obtained by connecting a plurality of the second trapping unit to one another.

According to the invention, there is a disease determination supporting apparatus comprising: the above apparatus; and a unit, which supports determination of a possibility of disease of the subject depending on a component and concentration of the analyzed first gas component.

### BRIEF DESCRIPTION OF THE DRAWINGS

FIG. 1 is a conceptual diagram illustrating a principle of a method and apparatus for analyzing a gas component derived from living body according to the present invention.

FIGS. 2A and 2B are views illustrating an atmosphere defecating step by a defecating and concentrating apparatus of FIG. 1, and a gas component analyzing step which is performed after concentration of the living-body derived gas component contained in the expired gas of the subject.

FIG. 3 is a view showing results (chromatogram) of an analysis in which the atmosphere is not defecated but concentrated.

FIG. 4 is a view showing a chromatogram of the atmosphere which is defecated and concentrated.

FIG. 5 is a view showing a chromatogram of the expired air in respiration which is performed by the subject on the defecated atmosphere.

FIG. 6 is a view showing another embodiment of the invention.

FIG. 7 is a conceptual diagram of the apparatus of the invention for analyzing a living-body derived gas generated from a product substance of the subject such as blood or urine.

FIGS. 8A and 8B are views showing an example of analysis results of living-body derived gas components according to the invention.

FIG. 9 is a view showing the configuration of a related-art sampling apparatus for a living-body derived gas (expired gas).

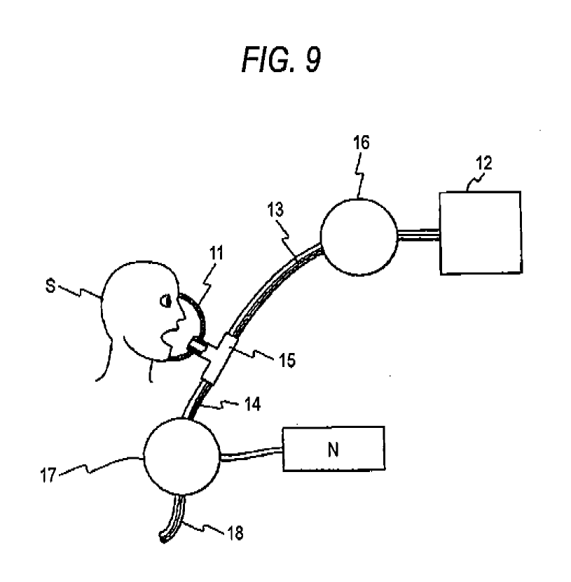

### DETAILED DESCRIPTION OF EMBODIMENTS

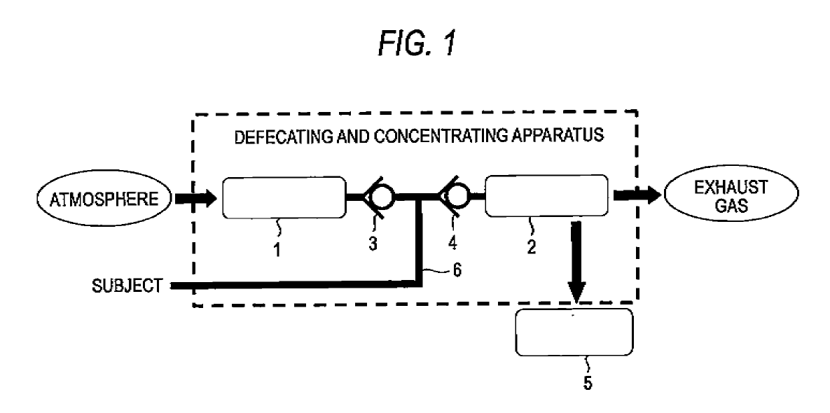

FIG. 1 is a conceptual diagram illustrating the principle of the method and apparatus for analyzing a gas component derived from living body according to the invention. Hereinafter, a gas component derived from living body is often referred to as a living-body derived gas component.

Referring to FIG. 1, 1 denotes a first trapping unit for trapping and removing impurities from an input gas to defecate the input gas, and 2 denotes a second trapping unit for, on the same principle as the first trapping unit 1, trapping and removing impurities from the input gas to extract the impurities.

Unidirectional valves 3, 4 are connected between the first trapping unit 1 and the second trapping unit 2. An expiration tube 6 extending toward a respiration mask (inspiration and exhaustion) for the subject (not shown) is connected between the unidirectional valves.

The exhaust air from the second trapping unit 2 is exhausted to the outside of the apparatus, and the impurities which are trapped by the second trapping unit 2 are analyzed by a gas analyzer 5 such as a gas chromatograph (GC).

In the invention, the portion configured by the first trapping unit 1, the second trapping unit 2, and the unidirectional valves 3, 4 shown in FIG. 1 is referred to as the defecating and concentrating apparatus.

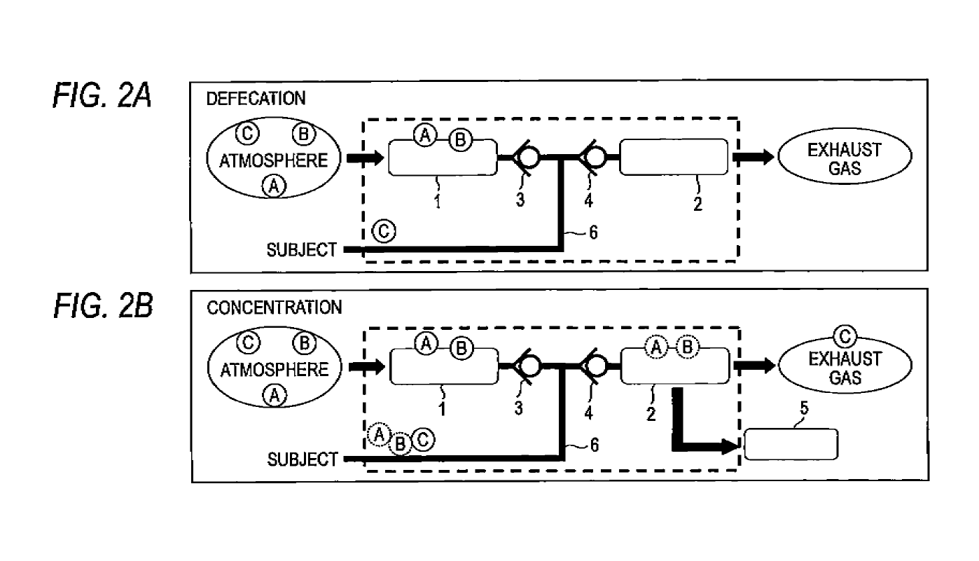

Next, an atmosphere defecating step by the defecating and concentrating apparatus of FIG. 1, and a gas component analyzing step which is performed after concentration of the living-body derived gas component contained in the expired gas of the subject will be described with reference to FIGS. 2A and 2B.

In FIGS. 2A and 2B, it is assumed that the atmosphere contains impurities A, B, and C, and the first trapping unit 1 and the second trapping unit 2 can trap the impurities A and B, but cannot trap the impurity C.

Although, in FIGS. 2A and 2B, the description is made under assumption that trappable impurities are A and B, and an untrappable impurity is C, the trappable impurities are not restricted to two kinds of A and B, and the untrappable impurity is not restricted to one kind of C. Each of the terms “impurities” and “impurity” means plural impurities which are determined by the principle of the corresponding trapping unit.

The trappable or untrappable impurities may vary depending on the principle of the trapping operation. Even in the case of a trappable impurity, moreover, it is not always required to trap 100% of the impurity, and a slight amount of the impurity may not be trapped and may be exhausted.

As the first trapping unit 1 and the second trapping unit 2, a unit is used which operates on a principle that, among living-body derived gases of the subject, can trap at least an object gas to be analyzed by the gas analyzer.

FIG. 2A shows a state where the subject performs inspiration and therefore the atmosphere which is defecated by the first trapping unit 1 is supplied through the unidirectional valve 3 to a respiratory mask of the subject which is not shown.

In this state, among the impurities A, 3, and C contained in the atmosphere, A and B are trapped by the first trapping unit 1, and the defecated atmosphere is supplied through the unidirectional valve 3 in order to be subjected to respiration of the subject, and then inhaled.

Then, FIG. 2B shows a state where the impurities A and B contained in the atmosphere are trapped by the first trapping unit 1, the defecated atmosphere which contains only the impurity C is inhaled by the subject, and then the expired gas is exhausted.

The expired gas of the subject contains the living-body derived gas components A and B (respectively enclosed by broken line circles). Therefore, the gas components are supplied together with the impurity C contained in the inspired gas, through the unidirectional valve 4 to the second trapping unit 2, the living-body derived gas components A and B are trapped, and the gas containing the remaining component C is exhausted to the outside of the apparatus.

The living-body derived gas components which are trapped by the second trapping unit 2 are given to the gas analyzer 5, and analysis of the living-body derived gas components is performed.

As described above, the atmosphere which is defecated by, as shown in FIG. 2A, trapping the impurities A and B contained in the atmosphere is supplied to the respiratory mask of the subject. Therefore, the gas analyzer can analyze the living-body derived gas components A and B contained in the expired gas of the subject, without being affected by the impurities contained in the atmosphere.

Next, the degree of defecation of the atmosphere by the first trapping unit 1 will be described.

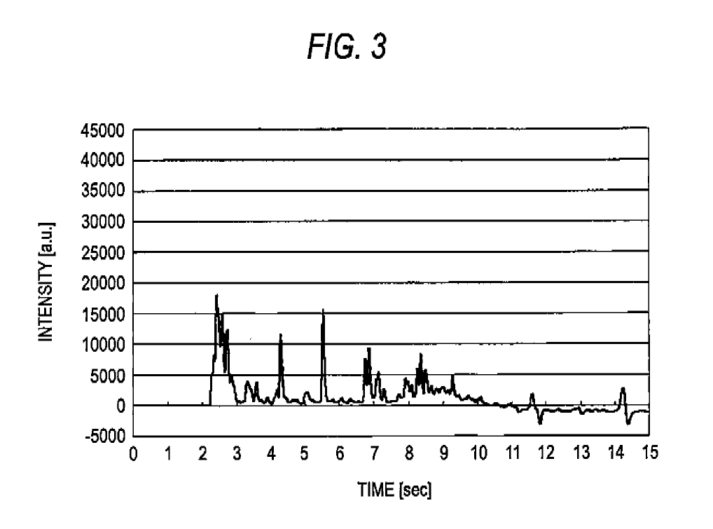

FIG. 3 is a view showing results (chromatogram) of an analysis in which, while the first trapping unit 1 is not disposed in FIG. 1 and the expiration tube 6 to the subject is blocked, the atmosphere is directly supplied to the second trapping unit 2, and components that are trapped by the second trapping unit are analyzed by the gas analyzer 5.

In FIG. 3, the abscissa indicates the time (sec), and the ordinate indicates the intensity (a.u.).

From FIG. 3, it is seen that various impurity components exist in a considerably large amount in the atmosphere, and affect as noises the measurement (analysis) of the living-body derived gas component.

The components shown in FIG. 3 include the impurities A and B shown in FIG. 2.

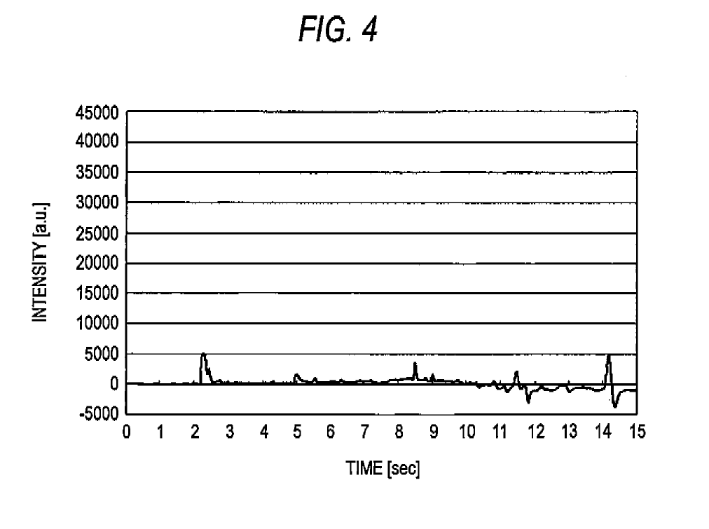

FIG. 4 is a view showing a chromatogram of an analysis in which, while the expiration tube 6 to the subject is blocked, the atmosphere is defecated by the first trapping unit 1, the defecated atmosphere is supplied to the second trapping unit 2, and components that are trapped by the second trapping unit 2 are analyzed by the gas analyzer 5.

Similarly with FIG. 3, in FIG. 4, the abscissa indicates the time (sec), and the ordinate indicates the intensity (a.u.).

From FIG. 4, it is seen that, in the atmosphere which is defecated by the first trapping unit 1, the intensities of impurity components are very lower than those of FIG. 3, and impurity components are contained only in amounts at which they do not affect as noises the measurement (analysis) of the living-body derived gas.

The components shown in FIG. 4 include impurities which, in the impurities A and B shown in FIG. 2, are not completely trapped by the first trapping unit 1.

Next, an example in which the expired gas of the subject is analyzed by the apparatus for analyzing a gas component derived from living body according to the invention and shown in FIG. 1 will be described.

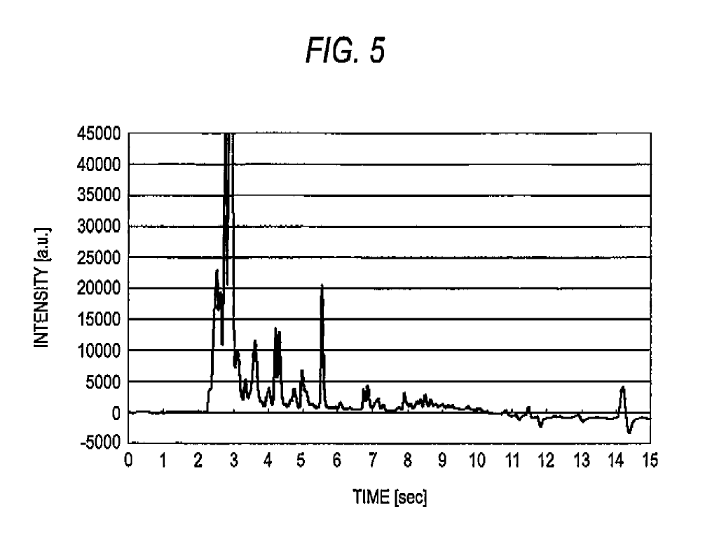

FIG. 5 is a view showing a chromatogram of the expired air in respiration which is performed by the subject on the atmosphere that is defecated by the first trapping unit 1.

Similarly with FIGS. 4 and 3, in FIG. 5, the abscissa indicates the time (sec), and the ordinate indicates the intensity (a.u.).

From FIG. 5, it is seen that the expired air of the subject contains A and B which are living-body derived gas components.

In the example of FIG. 5, the subject is a person who has a drinking habit. Therefore, the analysis results can be used in checking for drinking.

According to the invention, the components A and B which are trapped by the second trapping unit 2 are analyzed by the gas analyzer 5 (for example, a gas chromatograph), so that the analysis of the living-body derived gas components can be performed without being affected by impurities contained in the atmosphere.

In the case where a component D other than the impurities A and B which are contained in the atmosphere exists as a living-body derived gas component, the same components (A and B) as the above-described impurities can be trapped by the second trapping unit 2, but the component D can be trapped, or cannot be trapped and is exhausted depending on the principle and performance of the second trapping unit 2.

In the case where the component D can be trapped by the second trapping unit 2, the components A, B, and D which are trapped by the second trapping unit 2 are analyzed by the gas analyzer 5 (for example, a gas chromatograph), so that the analysis of the living-body derived gas components can be performed without being affected by impurities contained in the atmosphere.

In the case where the component D cannot be trapped by the second trapping unit 2, A and B which are trapped by the second trapping unit 2 can be analyzed by the gas analyzer 5 (for example, a gas chromatograph), but the component D cannot be analyzed.

The concentrated sample which is trapped by the second trapping unit is analyzed by the following apparatus including: Analyzer: GC-2014 (SHIMADZU CORPORATION); Column: G-100, Df5nm40m (Chemicals Evaluation and Research Institute); and Detector: hydrogen flame ionization detector (FID), and analysis conditions including: Carrier gas: He 25 ml/min; Sample amount: 1 ml; and Column temperature: 60° C., 3 min→+20° C./min→200° C., 5 min.

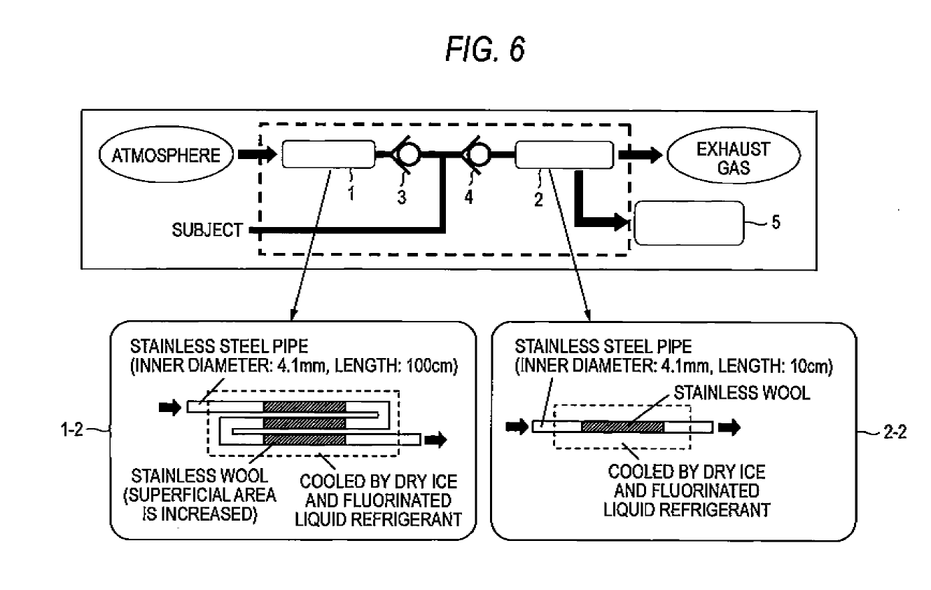

Next, another embodiment of the invention will be described with reference to FIG. 6.

The configuration shown in FIG. 6 is substantially identical with that shown in FIG. 1, but the configurations of the first and second trapping units are slightly different from each other.

The first trapping unit 1 and the second trapping unit 2 are identical with each other in principle of trapping of impurities, but different from each other in that the first trapping unit 1 is configured by connecting in series plural stages (in FIG. 6, ten stages) of the configuration of the second trapping unit 2.

The configuration where the first trapping unit 1 is configured by connecting in series plural stages of the configuration of the second trapping unit 2 can improve the function (performance) of trapping the impurities A and B contained in the atmosphere to enhance the degree of defecation of the defecated atmosphere which is to be supplied to the subject. Therefore, the influence of impurities in the atmosphere can be further reduced.

Hereinafter, FIG. 6 will be described in detail.

In FIG. 6, 2-2 denotes a detailed configuration example of the second trapping unit 2.

In the second trapping unit 2-2 of FIG. 6, a stainless steel pipe (inner diameter: 4.1 mm, length: 10 cm) filled with stainless wool is cooled by a combination of dry ice and a fluorinated liquid refrigerant.

In the first trapping unit 1-2, a stainless steel pipe (inner diameter: 4.1 mm, length: 100 cm) filled with stainless wool is cooled by a combination of dry ice and a fluorinated liquid refrigerant.

In the example of FIG. 6, the length of the stainless-wool filled portion of the first trapping unit is 10 times of that of the second trapping unit, and hence the superficial area of the portion is larger. Therefore, the trapping efficiency for impurities contained in the atmosphere is increased, and the degree of defecation of the atmosphere can be enhanced.

In the case where trapping units such as shown in FIG. 6 is used, after trapping, the second trapping unit 2 (concentrating portion) is closed, and then the sample is heated at about 80° C. for 30 minutes to obtain a concentrated sample, and analyzed by the gas analyzer 5 such as a gas chromatograph, thereby performing analysis of a living-body derived gas component without being affected by impurities existing in the atmosphere.

In the embodiment of FIG. 6, the first trapping unit 1 is configured by connecting in series a plurality of second trapping units 2. The first trapping unit may be configured in another manner without changing the principle of trapping of impurities by, for example, connecting not in series but in parallel plural second trapping units so that the trapping performance of the first trapping unit 1 is made higher than that of the second trapping unit 2.

In the above, the method and apparatus for analyzing a living-body derived gas contained in the expired gas of the subject have been described in detail. A living-body derived gas is generated also from blood, urine, skin or the like of the subject. Therefore, a method and apparatus for analyzing a living-body derived gas generated from such a substance will be described in detail.

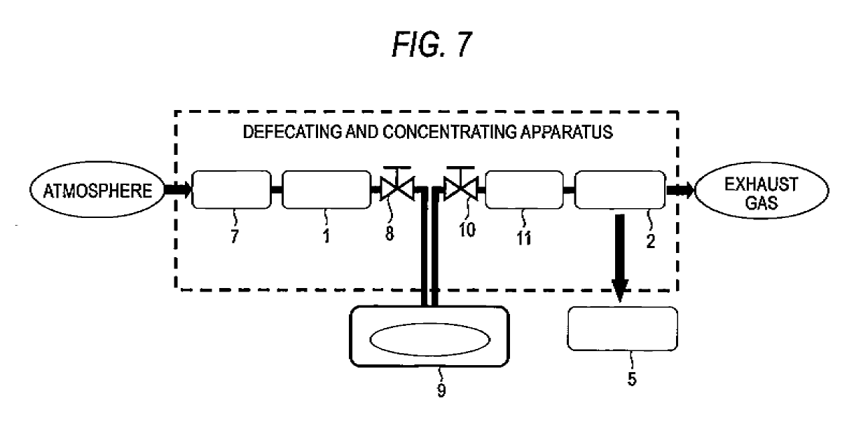

FIG. 7 is a conceptual diagram of the apparatus of the invention for analyzing a living-body derived gas generated from a product substance of the subject such as blood or urine.

In the figure, the components identical with the above-described components are denoted by the same reference numerals, and their description is omitted.

An exhaust port of a first pumping unit 7 in which an intake port is opened in the atmosphere communicates with a chamber 9 in which a product substance of the subject such as blood or urine, through the first trapping unit 1 and a first electromagnetic valve 8.

The chamber 9 communicates with the outside through a second electromagnetic valve 10, a second pumping unit 11, and the second trapping unit 2.

The first pumping unit 7 has a function of pressure feeding the atmosphere to the first trapping unit 1, and that which is defecated by the first trapping unit 1 to the chamber 9. The first electromagnetic valve 8 has a function of opening and shutting a path through which the first trapping unit 1 communicates with the chamber 9.

The second pumping unit 11 has a function of pressure feeding the atmosphere of the chamber 9 to the second trapping unit 2, and the second electromagnetic valve 10 has a function of opening and shutting a path through which the chamber 9 communicates with the second pumping unit 11.

The chamber 9 is configured by a flexible material. A product substance of the subject such as blood or urine is placed in the chamber so as to be exposed to the atmosphere which is defecated by the first trapping unit 1.

According to the configuration, in order to detect and analyze living-body derived gas components contained in the product substance of the subject such as blood or urine, the product substance of the subject is placed in the chamber 9, the first electromagnetic valve 8 is opened in accordance with instructions from a controller which is not shown, and the first pumping unit 7 is operated for a predetermined time period to feed a constant amount of the defecated atmosphere into the chamber 9.

Thereafter, the product substance of the subject is exposed to the defecated atmosphere under a state where the first electromagnetic valve 8 and the second electromagnetic valve 10 are closed.

Next, the second electromagnetic valve 10 is opened, and the second pumping unit 11 is operated to feed the atmosphere in the chamber 9 to the second trapping unit 2, thereby trapping the living-body derived gas components.

As described above, the chamber 9 is configured by a flexible material. As the atmosphere in the chamber 9 is further exhausted, therefore, the chamber 9 gradually contracts, so that the atmosphere in the chamber 9 is smoothly fed to the second trapping unit 2.

In a similar manner as described above, the living-body derived gas components which are trapped by the second trapping unit 2 are given to the gas analyzer 5, and analysis of the living-body derived gas components is performed.

In the case where the chamber 9 is configured by a rigid material, in the chamber 9, an inflow port for the defecated atmosphere and an outflow port to the second trapping unit 2 are separated from each other as far as possible.

The first electromagnetic valve 8 and the second electromagnetic valve 10 are closed, and the product substance of the subject is exposed to the defecated atmosphere for a predetermined time period. Thereafter, the first electromagnetic valve 8 and the second electromagnetic valve 10 are opened, and the second pumping unit 11 is operated to feed the atmosphere in the chamber 9 to the second trapping unit 2, thereby trapping the living-body derived gas components.

As described above, the atmosphere inflow port of the chamber 9 is separated from the outflow port. Therefore, the atmosphere in the chamber 9 is smoothly fed to the second trapping unit 2 while being pushed by the defecated atmosphere supplied from the first trapping unit 1.

In the case where living-body derived gas components generated from skin of, for example, a hand of the subject are to be detected and analyzed, a configuration is employed where the chamber 9 in FIG. 7 is configured by a flexible material, a hole through which a hand or the like can be inserted is disposed, and a hermetical seal can be made after insertion.

The detection and analysis of the living-body derived gas components are performed in a similar manner as the above-described case of blood, urine, or the like.

Next, an analysis example of analysis results of living-body derived gas components according to the invention will be described.

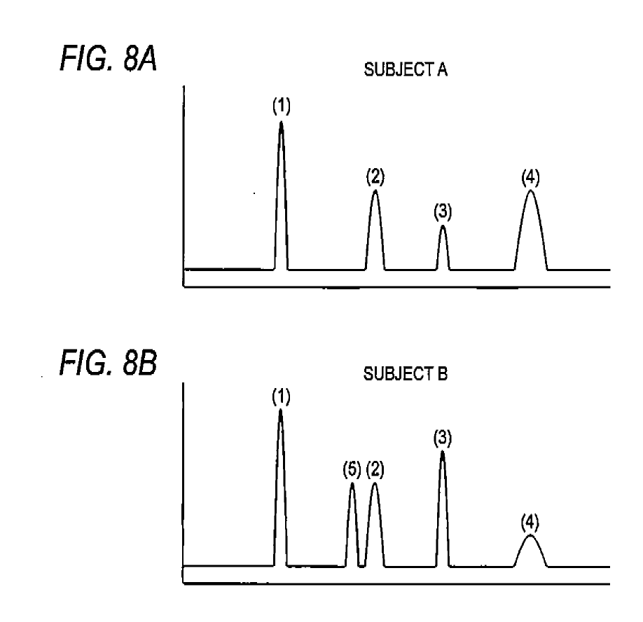

FIGS. 8A and 8B are views showing an example of analysis results of living-body derived gas components according to the invention. FIG. 8A shows analysis results of subject A, and FIG. 8B shows analysis results of subject B.

When the figures are compared to each other, the followings are seen.

Component (1) is a component common to subjects A and B.

Component (2) is a component common to subjects A and B.

Component (3) is a component the concentration of which is higher in subject B.

Component (4) is a component the concentration of which is lower in subject B.

Component (5) is a component which is detected only in subject B.

Components contained in the living-body derived gas, and their concentrations vary depending on the living habit and health condition of the subject. When such analysis results are accumulated and a determination is conducted based on the results and other inspection results, therefore, it is possible to obtain useful information concerning to healthcare of the subject and diagnosis of disease.

Examples of living-body derived gas components and diseases relating to the components are as follows.

### Example 1

### Acetone

In a diabetic patient, the acetone concentration in the expired gas is sometimes raised (because acetone is easily produced in the body when insulin secretion is reduced).

### Example 2

### Straight-Chain Hydrocarbon

There are reports that, in a lung cancer patient, the concentrations of (plural kinds of) straight-chain hydrocarbons in the expired gas show a distinctive pattern.

### Example 3

### Nitrogen Monoxide

It is said that nitrogen monoxide in the expired gas correlates with airway inflammatory disorder such as bronchial asthma (when a patient has the disorder, nitrogen monoxide is increased).

According to an aspect of the invention, it is possible to realize a method and apparatus for analyzing a gas component derived from living body which is less affected by the impurity removal performance of clean-air supplying unit (air defecating unit), and in which analysis of a gas component derived from living body Can be performed in a reduced number of steps.

What is claimed is:

1. A method of analyzing a gas component derived from living body, the method comprising;
extracting a first gas component from a gas contained in an atmosphere by a first method and removing the extracted first gas component from the gas to defecate the atmosphere;
obtaining a mixed gas of the defecated atmosphere and a second gas component from a subject;
extracting the first gas component from the mixed gas by a second method; and
analyzing the first gas component extracted from the mixed gas,
wherein the second method is the same as the first method.

2. An apparatus for analyzing a gas component derived from living body, the apparatus comprising;
a first trapping unit, which extracts a first gas component from a gas contained in an atmosphere by a first method, and removes the first gas component from the gas to defecate the atmosphere;
a mixing unit, which mixes the defecated atmosphere with a second gas component from a subject, to generate a mixed gas;
a second trapping unit, which extracts the first gas component from the mixed gas by a second method; and
a analyzing unit, which analyzes the first gas component extracted by the second trapping unit,
wherein the second method is the same as the first method.

3. The apparatus according to claim 2, wherein
the first trapping unit cools the gas contained in the atmosphere, which flows thereinto, to liquefy or solidify the gas, thereby extracting the first gas component, and
the second trapping unit cools the mixed gas, which flows thereinto, to liquefy or solidify the mixed gas, thereby extracting the first gas component.

4. The apparatus according to claim 2, wherein each of the first and second trapping units is provided with a material which sorbs the first gas component.

5. The apparatus according to claim 2, wherein the mixing unit generates the mixed gas by allowing the subject to breathe the atmosphere defecated by the first trapping unit.

6. The apparatus according to claim 2, wherein the mixing unit generates the mixed gas by exposing a product substance of the subject or a part of the subject to the atmosphere defecated by the first trapping unit.

7. The apparatus according to claim 2, wherein the first trapping unit includes a function which is obtained by connecting a plurality of the second trapping unit to one another.

8. A disease determination supporting apparatus comprising:
the apparatus according to claim 2; and
a unit, which supports determination of a possibility of disease of the subject depending on a component and concentration of the analyzed first gas component.
